(19) United States
(12) Patent Application Publication
(10) Pub. No.: US 2026/0243751 A1
(43) Pub. Date: Aug. 20, 2026

(54) NEGATIVE ION DETECTION DEVICE

(71) Applicant: MITSUBISHI HEAVY INDUSTRIES, LTD., Tokyo (JP)

(72) Inventors: Yuta Nakatsuchi, Tokyo (JP); Tetsuya Sawatsubashi, Tokyo (JP)

(73) Assignee: MITSUBISHI HEAVY INDUSTRIES, LTD., Tokyo (JP)

(21) Appl. No.: 19/473,296

(22) PCT Filed: Feb. 20, 2024
(86) PCT No.: PCT/JP2024/006000

§ 371 Date: Oct. 7, 2025

(30) Foreign Application Priority Data
May 30, 2023 (JP) 2023-088383

(51) Int. Cl. G01N 33/18; F22B 37/38

(52) U.S. Cl. CPC G01N 33/182; F22B 37/38

(57) ABSTRACT

An anion detection device includes: a supply pipe through which sample water containing plural kinds of ions is circulated; a preheater provided on the supply pipe and configured to preheat the sample water; a heating tank where the preheated sample water is further heated to separate the sample water into steam and drain water, the steam containing a substance derived from ions as a separation target among the plural kinds of ions; a discharge pipe configured to extract the drain water from the heating tank; an ion exchange unit provided on the discharge pipe and containing an ion exchange resin that removes the ions as the separation target remaining in the drain water; and a concentration detecting unit provided downstream of the ion exchange unit on the discharge pipe and configured to detect a concentration of anions in the drain water.

### TECHNICAL FIELD

The present disclosure relates to an anion detection device.

Priority is claimed on Japanese Patent Application No. 2023-088383, filed May 30, 2023, the content of which is incorporated herein by reference.

### BACKGROUND ART

In a water-steam cycle of a power plant, in order to prevent equipment or a pipe of a system from being damaged by corrosion, a component such as chloride ions or sulfate ions that causes corrosion is strictly managed to prevent ingress thereof. However, due to seawater leakage caused by damage of a cooling pipe of a condenser, a malfunction of a makeup water production device, or the like, the above-described impurity component may also ingress in the water-steam cycle. When the impurity ingresses, in order to minimize the range of contamination by the impurity and the effect of corrosion, it is necessary to immediately take a measure such as discharge of the impurity to the outside of the system using a system water blowdown, chemical addition for preventing a decrease in pH, plugging of a leakage occurrence pipe, or plant operation stop. To that end, it is important to promptly detect impurity ingress, and a cation conductivity is measured in most plants for monitoring the impurity in the water-steam cycle.

The cation conductivity is a method of measuring a conductivity after removing a cation component, for example, by allowing sample water collected from the water-steam cycle to pass through a cation exchange resin. A water treatment agent such as ammonia is added to the water-steam cycle to suppress corrosion. When a small amount of impurity ingresses, a water treatment agent concentration is much higher than an impurity concentration. Therefore, a change in conductivity caused by impurity ingress is much smaller than that in conductivity caused by the water treatment agent, and even when the conductivity is measured in this state, ingress of the impurity cannot be verified. Accordingly, an increase in conductivity caused by the water treatment agent is eliminated by removing the water treatment agent (ammonia or the like) using the cation exchange resin or the like, and high-sensitivity impurity ingress detection using a conductivity can be performed by exchanging counter ions of cations with H+ to increase the conductivity per impurity concentration.

As a specific example of this type of detection method and device, a technique described in PTL 1 below is known. PTL 1 below discloses a degassed cation conductivity meter as a method of removing carbon dioxide in feed water and steam to measure a cation conductivity. This method is a method of measuring a cation conductivity without being affected by dissolved gas by measuring the cation conductivity after performing a degassing pretreatment of a sample. In this method, a system capable of measuring a cation conductivity without being affected by carbon dioxide by heating a target sample to degas and remove carbon dioxide is used.

Here, recently, there has been a demand for increasing resistance to corrosion by setting the ammonia concentration injected into feed water to be higher than that in the related art. However, in the system in the related art, first, when the ammonia concentration in feed water is increased to remove ammonia using a cation exchange resin, a breakthrough time of the cation exchange resin is reduced, and an exchange frequency of the cation exchange resin increases. As a result, there is a concern that an increase in maintenance workload and an increase in device operating cost may be caused.

Accordingly, there is proposed a technique in which, by continuously supplying liquid sample water from above while introducing the sample water into a heating tank and producing steam in a lower portion of the tank, ammonia and carbon dioxide dissolved in the solution are recovered to the steam such that ammonia and carbon dioxide in the solution are removed with high efficiency. The liquid discharged as drain water from the heating tank contains only a small amount of ammonia ions. By allowing this drain water to pass through the ion exchange resin, the ammonia ions are removed.

### CITATION LIST

### Patent Literature

[PTL 1] Japanese Patent No. 6108021

### SUMMARY OF INVENTION

### Technical Problem

However, in the technique using the above-described heating tank, when the drain water is introduced to the outside, steam may ingress into a discharge pipe together with drain water. In this case, there is a problem in that ammonia and carbon dioxide in the steam of the sample water ingress into the drain water such that the concentrations of these components increase. As a result, the removal efficiency of the ammonia ions and the carbonate ions decreases, and the detection accuracy of nonvolatile ions such as chloride ions as a detection target decreases.

The present disclosure provides an anion detection device capable of more accurately detecting anions.

### Solution to Problem

An anion detection device according to the present disclosure includes: a supply pipe through which sample water containing plural kinds of ions is circulated; a preheater provided on the supply pipe and configured to preheat the sample water; a heating tank where the preheated sample water is further heated to separate the sample water into steam and drain water, the steam containing a substance derived from ions as a separation target among the plural kinds of ions; a discharge pipe configured to extract the drain water from the heating tank; an ion exchange unit provided on the discharge pipe and containing an ion exchange resin that removes the ions as the separation target remaining in the drain water; and a concentration detecting unit provided downstream of the ion exchange unit on the discharge pipe and configured to detect a concentration of anions in the drain water, in which the heating tank includes a tank main body, a supply portion provided above the tank main body and configured to supply the preheated sample water, a packing disposed inside the tank main body, a storage portion provided below the tank main body and configured to store the preheated sample water, a heating portion provided in the storage portion and configured to heat the sample water to produce steam, and a discharge portion configured to discharge the steam to an outside of the tank main body, and an inlet-side end portion of the discharge pipe is positioned lower than a liquid level in a steady state in the heating tank.

### Advantageous Effects of Invention

With the anion detection device according to the present disclosure, anions can be more accurately detected.

### BRIEF DESCRIPTION OF DRAWINGS

FIG. 1 is an overall view illustrating a configuration of an anion detection device according to an embodiment of the present disclosure.

FIG. 2 is an enlarged view of a main part illustrating a heating tank according to the embodiment of the present disclosure.

FIG. 3 is an enlarged view of a main part illustrating a first modification example of the heating tank according to the embodiment of the present disclosure.

FIG. 4 is an enlarged view of a main part illustrating a second modification example of the heating tank according to the embodiment of the present disclosure.

### DESCRIPTION OF EMBODIMENTS

### Configuration of Anion Detection Device

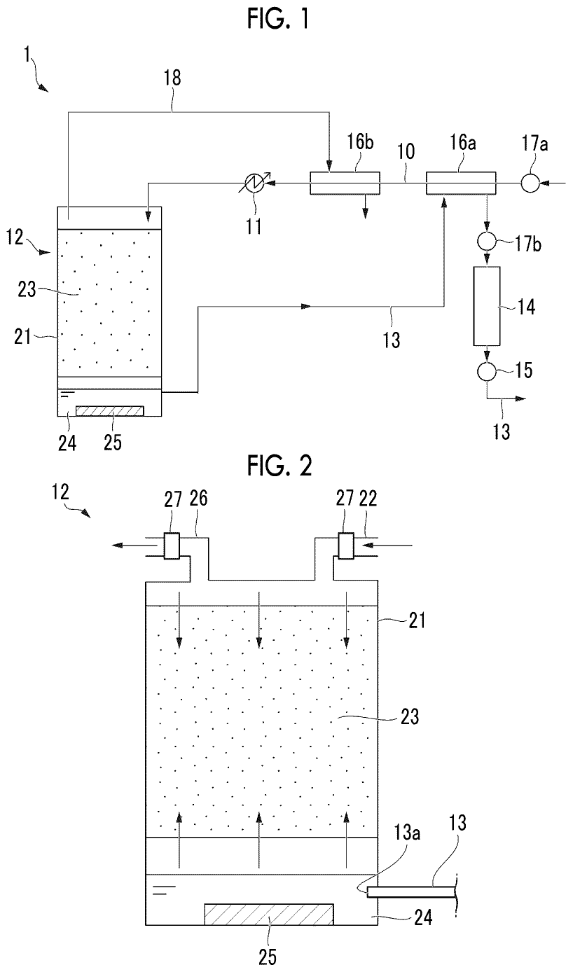

Hereinafter, an anion detection device 1 according to an embodiment of the present disclosure will be described with reference to FIGS. 1 and 2. This anion detection device 1 is a device for detecting and measuring concentrations of anions in feed water, condensed water, drum water, and steam of a steam turbine plant. In the steam turbine plant, a condenser for returning high-temperature steam to a liquid state is provided. The condenser is a heat exchanger that uses a refrigerant such as seawater to exchange heat between the seawater and steam. Here, when a refrigerant pipe in the condenser is damaged, there is a concern that the refrigerant such as seawater may be leaked to feed water such that pipes or various devices are damaged.

Accordingly, the anion detection device 1 is used for detecting anions containing a corrosion-causing component such as chloride ions (Cl⁻) that are anions as a detection target. On the other hand, in order to prevent corrosion of a pipe, a water treatment agent such as ammonia or hydrazine is injected into feed water. The anion detection device 1 detects and measures a concentration of anions such as chloride ions without being affected by these components.

As illustrated in FIG. 1, the anion detection device 1 includes a supply pipe 10, a preheater 11, a heating tank 12, a discharge pipe 13, an ion exchange unit 14, a concentration detecting unit 15, a first heat exchanger 16a, a second heat exchanger 16b, a first flowmeter 17a, a second flowmeter 17b, and a steam discharge pipe 18.

The supply pipe 10 is a pipe through which sample water collected from feed water, condensed water, drum water, steam, or the like of the steam turbine plant is circulated. The supply pipe 10 extends from an inlet that is an end portion on the upstream side to the heating tank 12. On the supply pipe 10, the first flowmeter 17a, the first heat exchanger 16a, the second heat exchanger 16b, and the preheater 11 are disposed in order from the upstream side to the downstream side.

The first flowmeter 17a measures a flow rate of the sample water flowing through the supply pipe 10, and transmits the measured flow rate to the outside as a numerical value. Although described below in detail, in the first heat exchanger 16a and the second heat exchanger 16b, heat exchange is performed between steam introduced from the heating tank 12 and drain water or the sample water. As a result, the sample water is heated to a high temperature. The preheater 11 further heats the sample water by supply of a heat medium supplied from the outside or using a heating mechanism such as an electric heater or a ceramic heater. More specifically, the preheater 11 heats the sample water until the sample water enters a state where a steam pressure thereof is the same as the atmospheric pressure or an internal pressure of the heating tank while being maintained in the liquid state. In the following description, the sample water in this state will also be simply referred to as “preheated sample water”.

Next, the configuration of the heating tank 12 will be described with reference to FIG. 2. The heating tank 12 includes a tank main body 21, a supply portion 22, a packing 23, a storage portion 24, a heating portion 25, a discharge portion 26, and a heating tank pressure regulating valve 27.

The tank main body 21 has a tubular shape extending in the vertical direction. “Tubular shape” described herein includes tubular shapes having a cross-sectional shape of a cylindrical shape, a square tubular shape, or a polygonal shape. The supply portion 22 connected to the above-described supply pipe 10 is provided above the tank main body 21. The preheated sample water flows into the tank main body 21 through the supply portion 22.

In the tank main body 21, the packing 23 is disposed. The packing 23 is provided for promoting mixing of fluid in the tank main body 21 and increasing the contact area. Specific examples of the packing 23 include packings having a shape called a Raschig ring or a Berl saddle. In addition, any substance can be used as long as it is used as a packing, that is, it has an action of increasing a gas-liquid contact area or promoting stirring.

The storage portion 24 is provided below the tank main body 21. The storage portion 24 is a container for storing the sample water flowing downward in the tank main body 21 through the supply portion 22. The heating portion 25 is provided in the storage portion 24. The heating portion 25 heats the preheated sample water, that is, applies latent heat to produce steam. As an example of the heating portion 25, specifically, an electric heater capable of adjusting a heating temperature is suitably used. In addition, a ceramic heater or the like can also be used as the heating portion 25. In addition, the sample water overflowing from the storage portion 24 is discharged to the outside through a pipe (not illustrated).

The discharge pipe 13 is connected to the storage portion 24. The discharge pipe 13 extends from the storage portion 24 to the concentration detecting unit 15. On the discharge pipe 13, the first heat exchanger 16a, the second flowmeter 17b, the ion exchange unit 14, and the concentration detecting unit 15 are disposed in this order. The sample water in the liquid state stored in the storage portion 24 flows downward in a pipeline (not illustrated) to be fed to the first heat exchanger 16a. In the first heat exchanger 16a, heat exchange is performed between the sample water flowing through the supply pipe 10 and the high-temperature sample water pressure-fed from the storage portion 24. As a result, the sample water flowing through the supply pipe 10 is heated, and the sample water flowing through the discharge pipe 13 is cooled.

An inlet-side end portion of the discharge pipe 13 is submerged in the liquid level of the heating tank 12. The height of the liquid level described herein refers to the height of the liquid level when contact between steam and the sample water described below in the heating tank 12 is regularly performed. Accordingly, only drain water flows into the discharge pipe 13, and the steam flowing above the liquid level does not flow into the discharge pipe 13.

The second flowmeter 17b measures the flow rate of the sample water flowing through the discharge pipe 13. The ion exchange unit 14 removes cations containing ammonia ions derived from the above-described water treatment agent among the ions in the sample water flowing through the discharge pipe 13 from the sample water using an ion exchange resin. That is, the sample water having passed through the ion exchange unit 14 substantially contains only nonvolatile anions (for example, chloride ions) as one detection target. This concentration detecting unit 15 detects and measures the concentration of the nonvolatile anions containing chloride ions, and transmits the measured concentration to the outside as a numerical value. The concentration detecting unit 15 is specifically a conductivity meter. Since the conductivity changes based on the concentration of the anions in the sample water, the concentration detecting unit 15 can finally acquire the concentration of the anions by measuring the conductivity.

The discharge portion 26 is provided above the tank main body 21. The steam produced by heating in the heating portion 25 is discharged from the discharge portion 26 provided above the tank main body 21. As illustrated in FIG. 1, the steam discharge pipe 18 extends between the discharge portion 26 and the second heat exchanger 16b. The steam discharged from the discharge portion 26 flows into the second heat exchanger 16b through the steam discharge pipe 18. In the second heat exchanger 16b, heat exchange is performed between the steam and the sample water flowing through the inside of the supply pipe 10. As a result, the sample water is heated, and the steam enters a liquid state or a gas-liquid mixed state and is discharged to the outside.

Further, the heating tank pressure regulating valve 27 is provided in each of the supply portion 22 and the discharge portion 26. The heating tank pressure regulating valve 27 is provided for adjusting an internal pressure of the tank main body 21. By changing the opening degree of the heating tank pressure regulating valve 27, the internal pressure of the tank main body 21 can be increased. Since the internal pressure of the tank main body 21 is high, the drain water introduced from the tank main body 21 to the outside is delivered under a pressure (pressure-fed).

Next, the operation of the anion detection device 1 will be described. During the operation of the anion detection device 1, first, the sample water is circulated to the supply pipe 10. At this time, it is assumed that the sample water contains not only the chloride ions as the detection target but also the ammonia ions derived from the water treatment agent and carbonate ions derived from carbon dioxide in the atmosphere. While being circulated in the supply pipe 10, the sample water passes through the first heat exchanger 16a, the second heat exchanger 16b, and the preheater 11 such that, as described above, the entirety or most of the sample water enters a state where the steam pressure thereof is the same as the atmospheric pressure or the internal pressure of the heating tank 12 while being maintained in the liquid state.

The preheated sample water flows into the heating tank 12 through the supply portion 22. In the tank main body 21 of the heating tank 12, the sample water flows to the downward storage portion 24 along the surface of the packing 23 while being dispersed such that the gas-liquid contact area increases. The sample water that reaches the storage portion 24 is heated by the heating portion 25 (latent heat is applied thereto) to produce steam. At this time, the steam does not contain ammonia and carbon dioxide, or contains ammonia and carbon dioxide at a very small concentration.

This steam flows upward in the tank main body 21. During this time, the steam comes into contact with the sample water in the liquid state flowing from above. At this time, the ammonia ions and the carbonate ions in the sample water in the liquid state move to the steam based on the double film theory. That is, the sample water in the liquid state and the steam are in contact with each other for a long time from directions opposite to each other as they flow downward. The concentrations of the ammonia ions and the carbonate ions in the sample water decrease toward the lower side. By repeating this cycle, a steady state is obtained. In the steady state, the sample water (drain water) in the liquid state stored in the storage portion 24 contains only a very small amount of ammonia ions.

This drain water flows into the ion exchange unit 14 after passing through the first heat exchanger 16a and the second flowmeter 17b through the discharge pipe 13. In the ion exchange unit 14, the ammonia ions in the drain water are removed due to the action of the ion exchange resin. That is, the drain water having passed through the ion exchange unit 14 contains only the nonvolatile anions containing the chloride ions as the detection target. Next, the nonvolatile anion concentration in the drain water is detected and measured by the conductivity meter as the concentration detecting unit 15.

On the other hand, the steam in contact with the sample water that is preheated while flowing upward in the tank main body 21 contains ammonia and carbon dioxide. This steam is fed to the second heat exchanger 16b through the steam discharge pipe 18, enters a liquid state or a gas-liquid mixed state, and is discharged to the outside.

As described above, with the above-described configuration, after being preheated by the preheater 11, the entirety or most of the sample water enters a state where a steam pressure thereof is the same as the atmospheric pressure while being maintained in the liquid state. Next, the sample water moved downward in the tank main body 21 through a gap of the packing 23 comes into contact with the heating portion 25 in the storage portion 24 to produce steam, that is, to enter a gas state. At this time, the concentrations of ammonia and carbon dioxide in the steam are lower than the concentrations of ammonia and carbon dioxide in the sample water (drain water) in the liquid state stored in the storage portion 24. When this steam moves upward in the tank main body 21, the steam comes into contact with new sample water flowing from above. At this time, based on the difference in the concentrations of ammonia and carbon dioxide between both the steam and the sample water, ammonia and carbon dioxide move from the sample water in the liquid state to the sample water in the gas state (steam). That is, the concentrations of ammonia and carbon dioxide in the sample water in the liquid state decrease toward the lower side. In particular, as described above, the sample water and the steam come into contact with each other from directions opposite to each other. Therefore, the difference in the concentrations of the ammonia and carbon dioxide between the sample water and the steam can be maintained to be large in the vertical direction in the tank main body 21. Therefore, the movement of the substance based on the double film theory can be promoted.

By continuously performing this cycle, the state where the concentrations of ammonia and carbon dioxide in the drain water stored in the storage portion 24 are low is maintained. Next, ammonia as a separation target in the drain water is removed by the ion exchange unit 14. Next, in the concentration detecting unit 15, the concentration of the nonvolatile anions (for example, the chloride ions) as the detection target is detected. This way, in a state where ammonia as the separation target and carbon dioxide are removed in advance, only the concentration of the nonvolatile anions as the detection target can be accurately measured. As a result, for example, during the operation of the steam turbine plant, whether or not foreign matter such as seawater ingresses into feed water can be instantly and accurately detected. Accordingly, the steam turbine plant can be more stably and smoothly operated.

### Actions and Effects

Here, when the discharge pipe 13 is exposed to the liquid level during the operation of the above-described heating tank 12, there is a concern that the steam produced from the sample water, that is, the steam containing ammonia and carbon dioxide may flow into the discharge pipe 13. When the steam flows into the discharge pipe 13, there is a problem in that ammonia ions and carbonate ions in the steam ingress into the drain water, and the concentrations of the components increase. As a result, there is a problem in that the removal efficiency of the ammonia ions and the carbonate ions decreases, and the detection accuracy of nonvolatile ions such as chloride ions as a detection target decreases. Accordingly, each of the above-described configurations is adopted in the present embodiment.

With the above-described configuration, the inlet-side end portion 13a of the discharge pipe 13 is positioned lower than the liquid level of the heating tank 12. That is, the inlet-side end portion 13a normally is submerged in the liquid level. Therefore, the steam flowing above the liquid level does not flow into the discharge pipe 13. As a result, an increase in the concentration of ammonia ions or carbonate ions in the drain water caused by the ingress of the steam in the discharge pipe 13 can be avoided. As a result, the drain water contains only the nonvolatile ions (for example, the chloride ions) as the detection target. Therefore, the chloride ions can be accurately detected. Accordingly, the ingress of the chloride ions or the like that adversely affect the operation of the plant can be discovered at an early stage. Therefore, a more stable operation of the plant can be realized.

In addition, with the above-described configuration, the heating tank pressure regulating valve 27 is provided in each of the supply portion 22 and the discharge portion 26 of the heating tank 12. By appropriately changing the opening degrees of the heating tank pressure regulating valves 27, the internal pressure of the heating tank 12 (tank main body 21) can be increased. As a result, the drain water overflowing from the heating tank 12 can be pressure-fed to the outside. Accordingly, the degree of freedom for a layout of a flow channel of the drain water can be further increased. Specifically, the drain water can be delivered due to the downward flow caused by the own weight, and the drain water can also be pressure-fed upward. As a result, a reduction in the size of the device and a reduction in manufacturing cost and maintenance cost caused by a reduction in the number of components can be realized.

### Other Embodiments

Above, the embodiments of the present disclosure have been described in detail with reference to the drawings, but the specific configuration is not limited to the embodiments, and includes design changes and the like within a scope not departing from the gist of the present disclosure.

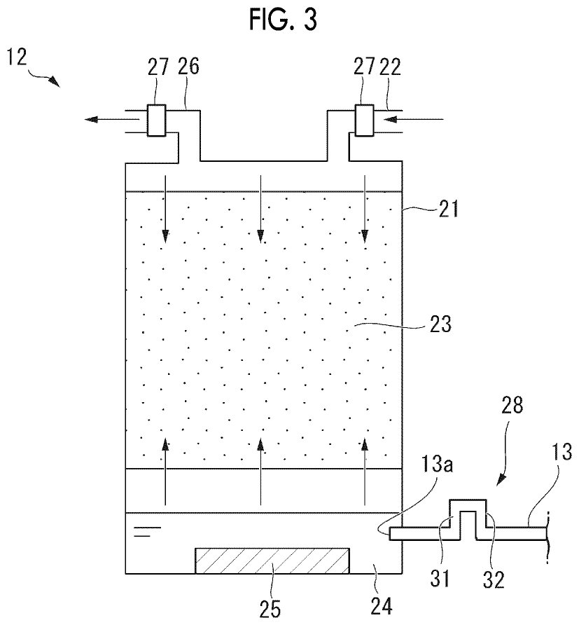

For example, the aspect of the discharge pipe 13 is not limited to the above-described embodiments, and a configuration illustrated in FIG. 3 can be adopted as a first modification example. In the example of the same drawing, a rising portion 28 is provided in the middle of the discharge pipe 13. The rising portion 28 includes a first section 31 that extends upward away from the inlet-side end portion 13a to the downstream side and a second section 32 that communicates with the first section 31 and extends downward away from the first section 31 to the downstream side. That is, a connecting portion between the first section 31 and the second section 32 is positioned higher than the inlet-side end portion 13a. With this configuration, the drain water is not discharged from the discharge pipe 13 until the liquid level of the heating tank 12 reaches the height of the rising portion 28. Accordingly, the steam flowing above the liquid level does not flow into the discharge pipe 13. As a result, an increase in the concentration of ammonia ions or carbonate ions in the drain water caused by the ingress of the steam in the discharge pipe 13 can be avoided. As a result, the drain water contains only the nonvolatile ions (for example, the chloride ions) as the detection target. Therefore, the chloride ions can be accurately detected. Accordingly, the ingress of the chloride ions or the like that adversely affect the operation of the plant can be discovered at an early stage. Therefore, a more stable operation of the plant can be realized.

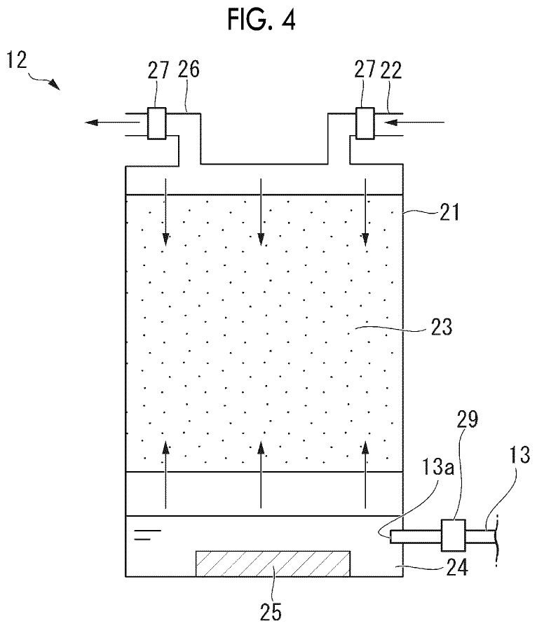

Further, a configuration illustrated in FIG. 4 can also be adopted as a second modification example. In the example of the same drawing, a pressure regulating valve 29 is provided on the discharge pipe 13. By changing the opening degree, the pressure regulating valve 29 can cause a pressure loss in the drain water flowing through the discharge pipe 13. With this configuration, the liquid level height of the heating tank 12 tends to increase by the amount of the pressure loss by the pressure regulating valve 29. That is, the inlet-side end portion of the discharge pipe 13 is likely to be submerged in the liquid level. Accordingly, the steam flowing above the liquid level does not flow into the discharge pipe 13. As a result, an increase in the concentration of ammonia ions or carbonate ions in the drain water caused by the ingress of the steam in the discharge pipe 13 can be avoided. As a result, the drain water contains only the nonvolatile ions (for example, the chloride ions) as the detection target. Therefore, the chloride ions can be accurately detected. Accordingly, the ingress of the chloride ions or the like that adversely affect the operation of the plant can be discovered at an early stage. Therefore, a more stable operation of the plant can be realized.

The above-described heating tank pressure regulating valve 27 is not necessarily provided. In addition, a configuration not including the first heat exchanger 16a and the second heat exchanger 16b can also be adopted. The order of the arrangement of these components may also be changed. With this configuration as well, the same actions and effects as described above can be obtained from the viewpoint of detecting anions.

### <Supplementary Notes>

The anion detection device described in each embodiment is understood as follows, for example.

(1) An anion detection device 1 according to a first aspect includes: a supply pipe 10 through which sample water containing plural kinds of ions is circulated; a preheater 11 provided on the supply pipe 10 and configured to preheat the sample water; a heating tank 12 where the preheated sample water is further heated to separate the sample water into steam and drain water, the steam containing a substance derived from ions as a separation target among the plural kinds of ions; a discharge pipe 13 configured to extract the drain water from the heating tank 12; an ion exchange unit 14 provided on the discharge pipe 13 and containing an ion exchange resin that removes the ions as the separation target remaining in the drain water; and a concentration detecting unit 15 provided downstream of the ion exchange unit 14 on the discharge pipe 13 and configured to detect a concentration of anions in the drain water, in which the heating tank 12 includes a tank main body 21, a supply portion 22 provided above the tank main body 21 and configured to supply the preheated sample water, a packing 23 disposed inside the tank main body 21, a storage portion 24 provided below the tank main body 21 and configured to store the preheated sample water, a heating portion 25 provided in the storage portion 24 and configured to heat the sample water to produce steam, and a discharge portion 26 configured to discharge the steam to an outside of the tank main body 21, and an inlet-side end portion 13a of the discharge pipe 13 is positioned lower than a liquid level in a steady state in the heating tank 12.

With the above-described configuration, the inlet-side end portion 13a of the discharge pipe 13 is positioned lower than the liquid level of the heating tank 12. That is, the inlet-side end portion 13a normally is submerged in the liquid level. Therefore, the steam flowing above the liquid level does not flow into the discharge pipe 13. As a result, an increase in the concentration of ammonia ions or carbonate ions in the drain water caused by the ingress of the steam in the discharge pipe 13 can be avoided.

(2) According to a second aspect, in the anion detection device 1 according to (1), the discharge pipe 13 includes a first section 31 that communicates with the heating tank 12 and extends upward away from the heating tank 12 to a downstream side, and a second section 32 that communicates with the first section 31 and extends downward away from the first section 31 to a downstream side.

With the above-described configuration, the discharge pipe 13 includes the first section 31 and the second section 32. The pipeline rises upward from the first section 31 to the second section 32. As a result, the drain water is not discharged from the discharge pipe 13 until the liquid level of the heating tank 12 reaches the height of the rising portion. Accordingly, the steam flowing above the liquid level does not flow into the discharge pipe 13. As a result, an increase in the concentration of ammonia ions or carbonate ions in the drain water caused by the ingress of the steam in the discharge pipe can be avoided.

(3) According to a third aspect, the anion detection device 1 according to (1) or (2), further includes a pressure regulating valve 29 provided on the discharge pipe 13.

With the above-described configuration, the pressure regulating valve 29 is provided on the discharge pipe 13. Therefore, the liquid level height of the heating tank 12 tends to increase by the amount of the pressure loss by the pressure regulating valve 29. That is, the inlet-side end portion 13a of the discharge pipe 13 is likely to be submerged in the liquid level. Accordingly, the steam flowing above the liquid level does not flow into the discharge pipe 13. As a result, an increase in the concentration of ammonia ions or carbonate ions in the drain water caused by the ingress of the steam in the discharge pipe 13 can be avoided.

(4) According to a fourth aspect, the anion detection device 1 according to any one of (1) to (3), further includes a heating tank pressure regulating valve 27 provided in each of the supply portion 22 and the discharge portion 26.

With the above-described configuration, the heating tank pressure regulating valve 27 is provided in each of the supply portion 22 and the discharge portion 26 of the heating tank 12. By appropriately changing the opening degrees of the heating tank pressure regulating valves 27, the internal pressure of the heating tank 12 can be increased. As a result, the drain water overflowing from the heating tank 12 can be pressure-fed to the outside. Accordingly, the degree of freedom for a layout of a flow channel of the drain water can be further increased.

### Industrial Applicability

With the anion detection device according to the present disclosure, anions can be more accurately detected.

### REFERENCE SIGNS LIST

1: anion detection device
10: supply pipe
11: preheater
12: heating tank
13: discharge pipe
14: ion exchange unit
15: concentration detecting unit
16a: first heat exchanger
16b: second heat exchanger
17a: first flowmeter
17b: second flowmeter
18: steam discharge pipe
21: tank main body
22: supply portion
23: packing
24: storage portion
25: heating portion
26: discharge portion
27: heating tank pressure regulating valve
28: rising portion
29: pressure regulating valve
31: first section
32: second section

1. An anion detection device comprising:
a supply pipe through which sample water containing plural kinds of ions is circulated;
a preheater provided on the supply pipe and configured to preheat the sample water;
a heating tank where the preheated sample water is further heated to separate the sample water into steam and drain water, the steam containing a substance derived from ions as a separation target among the plural kinds of ions;
a discharge pipe configured to extract the drain water from the heating tank;
an ion exchange unit provided on the discharge pipe and containing an ion exchange resin that removes the ions as the separation target remaining in the drain water; and
a concentration detecting unit provided downstream of the ion exchange unit on the discharge pipe and configured to detect a concentration of anions in the drain water, wherein
the heating tank includes
a tank main body,
a supply portion provided above the tank main body and configured to supply the preheated sample water,
a packing disposed inside the tank main body,
a storage portion provided below the tank main body and configured to store the preheated sample water,
a heating portion provided in the storage portion and configured to heat the sample water to produce steam, and
a discharge portion configured to discharge the steam to an outside of the tank main body, and
an inlet-side end portion of the discharge pipe is positioned lower than a liquid level in a steady state in the heating tank.

2. The anion detection device according to claim 1, wherein
the discharge pipe includes
a first section that communicates with the heating tank and extends upward away from the heating tank to a downstream side, and
a second section that communicates with the first section and extends downward away from the first section to a downstream side.

3. The anion detection device according to claim 1, further comprising a pressure regulating valve provided on the discharge pipe.

4. The anion detection device according to claim 1, further comprising a heating tank pressure regulating valve provided in each of the supply portion and the discharge portion.
